SEQUENCE LISTING (11 sequences)

SEQ ID NO: 1 - protein, 5 aa, Artificial
  Synthetic peptide
CLPRC

SEQ ID NO: 2 - protein, 14 aa, Artificial
  Synthetic peptide
KLAKLAKKLAKLAK

SEQ ID NO: 3 - protein, 14 aa, Artificial
  Synthetic peptide
KLAKKLAKLAKKLA

SEQ ID NO: 4 - protein, 14 aa, Artificial
  Synthetic peptide
KAAKKAAKAAKKAA

SEQ ID NO: 5 - protein, 21 aa, Artificial
  Synthetic peptide
KLGKKLGKLGKKLGKLGKKLG

SEQ ID NO: 6 - protein, 5 aa, Artificial
  Synthetic peptide
CAPAC

SEQ ID NO: 7 - protein, 5 aa, Artificial
  Synthetic peptide
CLPRC

SEQ ID NO: 8 - protein, 8 aa, Artificial
  Synthetic peptide
CPQPRPLC

SEQ ID NO: 9 - protein, 25 aa, Artificial
  Synthetic peptide
CKGGRAKDCGGKLAKLAKKLAKLAK

SEQ ID NO: 10 - protein, 30 aa, Artificial
  Synthetic peptide
CLPRCCKGGRAKDCGGKLAKLAKKLAKLAK

SEQ ID NO: 11 - protein, 9 aa, Artificial
  Synthetic peptide
CKGGRAKDC

(19) United States
(12) Patent Application Publication
(10) Pub. No.: US 2012/0028880 A1
(43) Pub. Date: Feb. 2, 2012

(54) VEGFR-1/NRP-1 TARGETING PEPTIDES

(76) Inventors: Renata Pasqualini, Houston, TX (US); Wadih Arap, Houston, TX (US); Ricardo Giordano, Sao Paulo (BR); Marina Cardó-Vila, Houston, TX (US); Ana Paula Valente, Rio de Janeiro (BR); Fabio Ceneviva Lacerda De Almeida, Rio de Janeiro (BR)

(21) Appl. No.: 12/672,647

(22) PCT Filed: Aug. 8, 2008
(86) PCT No.: PCT/US08/72675

§ 371 Date: Oct. 10, 2011

Related U.S. Application Data
(60) Provisional application No. 60/954,750, filed on Aug. 8, 2007.

(51) Int. Cl. A61K 38/02; C07K 7/06; C07K 7/64; C07K 19/00; C07K 5/083; C12N 7/00; C12N 1/20; C12N 1/16; C12N 5/071; C07K 17/00; C12N 9/96; A61P 11/06; A61P 35/02; A61P 35/00; A61P 19/02; A61P 3/04; A61P 3/10; A61P 27/02; A61P 19/08; A61P 9/10; A61P 17/06; A61P 27/06; C12Q 1/02; C07K 4/00

(52) U.S. Cl. 514/1.7; 530/300; 530/330; 530/321; 530/350; 530/331; 530/387.3; 530/380; 435/235.1; 435/252.1; 435/255.1; 435/325; 435/188; 435/375; 514/13.3; 514/6.9; 530/351; 514/1.1; 435/29

(57) ABSTRACT

The present invention concerns the fields of molecular medicine and targeted delivery of therapeutic agents. More specifically, the present invention relates to the identification of novel peptide sequences that incorporate the amino acids Leu-Pro-Arg (LPR), and particularly _{D}(LPR), that selectively target VEGFR-I and NRP-I expressing cells. Targeted molecules in accor-dance with the invention are useful in the treatment and detection of neovascular or angiogenic VEGF associated disorders, including but not limited to cancer, obesity, diabetes, asthma, arthritis, cirrhosis and ocular diseases.

The present application claims priority to U.S. Provisional Patent Application Ser. No. 60/954,750, filed on Aug. 8, 2007, which is hereby incorporated by reference in its entirety.

This invention was made with U.S. government support under grants CA103056 and CA100632 from the National Institutes of Health. The U.S. government therefore has certain rights in the present invention.

### BACKGROUND OF THE INVENTION

1. Field of the Invention

The present invention concerns the fields of molecular medicine and targeted delivery of therapeutic agents. More specifically, the present invention relates to the identification of novel peptide sequences that selectively target VEGFR-1 and NRP-1 as a therapeutic target for the treatment and detection of neovascular or angiogenic VEGF associated disorders, including but not limited to cancer, obesity, diabetes, asthma, arthritis, cirrhosis and ocular diseases.

2. Description of the Related Art

Blood vessels are essential bodily components that deliver oxygen and nutrients to almost all organs and tissues. Most vessels are formed during embryonic development, and in adults the formation of new blood vessels (a process called angiogenesis) is limited, mainly during wound healing and the normal female reproductive cycle. This creates an opportunity for therapy, as several diseases can progress only if they induce the formation of new blood vessels; cancer, obesity, diabetes, asthma, arthritis, cirrhosis and ocular diseases are among the many illnesses, likely to be slowed down or blocked by the development of angiogenesis inhibitors.

Vascular Endothelial Growth Factor (VEGF) is recognized as a central molecular control factor in angiogenesis, and three anti-VEGF drugs have now been approved by the U.S. Food and Drug Administration for treatment of specific types of cancer, with good but not ideal therapeutic effects (Kamba and McDonald, 2007). Therefore, the development of a new generation of drugs targeting the VEGF pathway is likely to have a significant impact in the therapeutic regiment for several diseases. VEGF is a key regulator of angiogenesis and stimulates endothelial cell division and migration by binding to cell surface VEGF tyrosine kinase receptors (VEGFR-1 and -2) and to neuropilins (NRP). Because VEGFR-2 is the main mediator of the VEGF mitogenic intracellular signaling, most drugs in the clinic today are aimed directly or indirectly at this specific receptor. On the other hand, VEGFR-1 and NRP-1 were initially believed to be either a decoy or a sink for VEGF (VEGFR-1) or a modulator of VEGFR-2 activity (NRP-1). However, research generated in the past few years suggests otherwise. Both receptors have a prominent role in angiogenesis (Carmeliet et al., 2001; Autiero et al., 2003; Luttun et al., 2004; Kaplan et al., 2005; Wu et al., 2006; Pan et al., 2007) and are important targets for angiogenesis therapy. For instance, monoclonal antibodies directed against VEGFR-1 and NRP-1 have shown promising results as anti-tumor agents, especially in combination with chemotherapeutics (Wu et al., 2006; Pan et al., 2007).

Many anti-VEGF drugs, such as bevacizumab (Avastin®) and ranibizumab (Lucentis®), are in clinical use and have shown some degree of efficacy in the treatment and management of neovascular disorders, including various types of cancers as well as neovascularization conditions affecting the eye, such as age-related macular degeneration. Unfortunately, such non-specific VEGF therapies have been demonstrated to have potentially serious side effects, including, in particular, heart related toxicities (e.g., chest pain, strokes, ministrokes, congestive heart failure and hear attacks, hemorrhage, proteinuria, hypertension, congestive heart failure, arterial thromboembolia, and gastrointestinal perforation). Studies have linked such side effects to the fact that drugs of this class, i.e., drugs that target vescular endothelial growth factor (VEGF-A; VEGF₁₆₅) directly, as opposed to selectively targeting receptors, can adversely effect normal VEGF pathways (Betsholtz et al., 2006). VEGF-A holds an exceptional position among the many molecules implicated in the regulation of blood vessel formation. During embryonic development, it controls a large number of processes, spanning from the expansion of the earliest cell progenitors of the vasculature to the control of proliferation and migration of endothelial cells, vessel remodeling, and arteriovenous specification (Ferrara, 2004). A correct level of VEGF-A protein is absolutely critical for vessel development, because a reduction of expression by half or an increase by two-fold are both fatal conditions for a mouse embryo.

The toxicities associated with VEGF-directed therapies are thought to be linked to the depriving of normal capillary beds and heart cells with VEGF that is needed for normal cell function and vascularization. Interestingly, the VEGF-dependent capillary beds share common features in that they exhibit high expression of VEGF receptors known as the type 2 and type 3 receptors (VEGFR-2 and VEGFR-3, respectively) and yet exhibit little or no VEGFR-1 receptors. However, VEGFR-1 receptors are found to be expressed in those tissues associated with desirable disease targets, including retinal vessels and tumor vasculature. Thus, it would be highly desirable to develop anti-VEGF therapeutics that specifically target VEGFR-1 receptors, in that such therapeutics would potentially exhibit reduced toxicities while maintaining the desired therapeutic activity. The present invention is directed to providing peptide ligands that selectively target VEGFR-1.

### SUMMARY OF THE INVENTION

The present invention overcomes deficiencies in the prior art by providing methods and compositions for selectively targeting VEGFR-1 and NRP-1 (hereinafter referred to as “VEGFR-1/NRP-1”) through the use of the targeting motif LPR (Leu-Pro-Arg), and more preferably _{D}(LPR). Selective targeting of VEGFR-1/NRP-1 through the use of the LPR motif is useful, for example, in the treatment of cancer or other disease states associate with angiogenesis or vascular growth, such as obesity, diabetes, asthma, arthritis, cirrhosis and ocular diseases.

In certain embodiments, the invention thus concerns isolated LPR targeting peptides, that is, targeting peptides that include the contiguous LPR sequence within it structure, for example, positioned at the amino terminus or carboxy terminus of the peptide or internally. While positioning the LPR sequence at a terminus is believed to be the most preferred, it is contemplated that internal positioning of LPR will nonetheless provide VEGFR-1/NRP-1 targeting capability. For ease of preparation and handling, certain such embodiments of the invention are directed to isolated peptides of 10 amino acids or less in size, comprising at least the contiguous amino acid sequence Leu Pro Arg. For this reason as well, even shorter peptides, such as peptide of 7 or 5 amino acids or less in size, and even the LPR tripeptide per se will be even more preferred. Thus, targeting peptides of the present invention may comprise 3, 4, 5, 6, 7, 8, 9 or 10 amino acids, wherein the contiguous LPR sequence or that of SEQ ID NO:1 is positioned therein.

In still other particular embodiments, the inventors contemplate specific peptides incorporating the LPR sequence that are capable of being prepared in a cyclic form, such as peptide having a cysteine residue (“C”) at both termini, which may, where desired, be provided in cyclic form, such as through the formation of a di-cysteine (i.e., cystine). An example of such a peptide is Cys Leu Pro Arg Cys (SEQ ID NO:1). Such cyclic peptides may be of particular importance in that disulfide bonds in peptides makes them remarkably stable to chemical, thermal or enzymatic degradation. Such cyclic peptides may be of particular importance in therapeutic and diagnostic applications, where poor availability, susceptibility to proteolysis and short in vivo half-lives are concerned.

In still other embodiments, the invention contemplates the use of D amino acids for the preparation of all or part of the foregoing peptides. Peptides composed of D amino acids have certain advantages over those composed of L amino acids in that the use of D amino acids render the targeting peptides of the invention generally resistant to the effects of proteases and peptidases. Particularly preferred for such aspects of the invention are targeting peptides that consist entirely of D amino acids such as _{D}(Leu Pro Arg) and _{D}(Cys Leu Pro Arg Cys) (SEQ ID NO:1).

In certain embodiments, an LPR targeting moiety, such as set forth above, may be operatively conjugated to a second molecule or substance. In preferred embodiments, the attachment is a covalent attachment, as exemplified by chemical conjugate (e.g., formed through the use of a chemical linker) or fusion constructs (e.g., formed by fusing the underlying nucleic acid coding region for such a peptide fused in frame with a nucleic acid coding region coding for a desired protein or peptide that one desires to have targeted to VEGFR-1/NRP-1). In the case of targeted proteins or peptides, the targeting peptide may be positioned at or near the amino or carboxy terminus (i.e., within the first or last 20 amino acids) of the protein or peptide that one desires to so target.

In various selected embodiments, the second molecule or substance is a diagnostic agent, a drug, a chemotherapeutic agent, a radioisotope, an anti-angiogenic agent, a pro-apoptosis agent, a cytotoxic agent, a peptide, a protein, a hormone, a growth factor, a cytokine, an antibiotic, an antibody or fragment or single chain antibody, an imaging agent, a survival factor, an anti-apoptotic agent, a hormone antagonist or an antigen. These molecules or substances are representative only and virtually any molecule that may yield a therapeutic or diagnostic benefit for the treatment of cancer may be attached to an LPR targeting moiety and/or administered to a subject within the scope of the invention.

Accordingly, where the molecule to be targeted is a pro-apoptotic agent, exemplary agents include etoposide, ceramide sphingomyelin, Bcl-2, Bax, Bid, Bik, Bad, caspase-3, caspase-8, caspase-9, fas, fas ligand, fadd, fap-1, tradd, faf, rip, reaper, apoptin, interleukin-2 converting enzyme, annexin V, (KLAKLAK)₂ (SEQ ID NO:2); (KLAKKLA)₂ (SEQ ID NO:3); (KAAKKAA)₂ (SEQ ID NO:4); or (KLGKKLG)₃ (SEQ ID NO:5). It should be noted that as with all of the peptides of the present invention, sequences such as the foregoing can be provided in either D or L form. For example, for the proapoptic peptides (e.g., SEQ ID NOs 2-5), both the D and L forms are believed to have similar proapoptotic activity, with the D form having a substantially longer half-life due to their relative proteinase resistance. In some instances, though, the L form will, in practice, have advantages due to potentially reduced toxic side effects (due to their shorter half-life).

Moreover, in embodiments where the molecule to be targeted in an anti-angiogenic agent, exemplary agents include thrombospondin, an angiostatin such as angiostatin 5, angiotensin, laminin peptides, fibronectin peptides, plasminogen activator inhibitors, tissue metalloproteinase inhibitors, interferons, a cytokine such as interleukin 12, platelet factor 4, IP-10, Gro-β, 2-methoxyoestradiol, proliferin-related protein, carboxiamidotriazole, CM101, Marimastat, pentosan polysulphate, angiopoietin 2 (Regeneron), herbimycin A, PNU145156E, 16K prolactin fragment, Linomide, thalidomide, pentoxifylline, genistein, TNP-470, an endostatin such as endostatin XVII and XV, paclitaxel, docetaxel), polyamines, a proteasome inhibitor, a kinase inhibitor, a signaling peptide, accutin, cidofovir, vincristine, bleomycin, AGM-1470, minocycline, the C-terminal hemopexin domain of matrix metalloproteinase-2, the kringle 5 domain of human plasminogen, a fusion protein of endostatin and angiostatin, a fusion protein of endostatin and the kringle 5 domain of human plasminogen, the monokine-induced by interferon-gamma (Mig), a fusion protein of Mig and IP10, soluble FLT-1 (fins-like tyrosine kinase 1 receptor), kinase insert domain receptor (KDR), pigment epithelium-derived factor, interferon-alpha (interferons in line 4), a signaling inhibitor (SU5416, SU6668, Sugen, South San Francisco, Calif.).

In further preferred embodiments, where the targeted molecule is a cytokine, exemplary cytokines include interleukin 1 (IL-1), IL-2, IL-5, IL-10, IL-11, IL-12, IL-18, IL-24, interferon-γ (INF-γ), INF-α, INF-β, a tumor necrosis factor such as TNF-α, or GM-CSF (granulocyte macrophage colony stimulating factor).

The foregoing examples are representative only and are not intended to exclude other pro-apoptosis agents, anti-angiogenic agents or cytokines known in the art.

In other embodiments of the invention, the isolated peptide may be attached to a macromolecular complex. In preferred embodiments, the macromolecular complex is a virus, a bacteriophage, a bacterium, a liposome, a microparticle, a nanoparticle (e.g., a gold nanoparticle), a magnetic bead, a yeast cell, a mammalian cell, or a bacterial cell. 1 the case of viruses, particularly preferred include a bacteriophage, lentivirus, papovavirus, adenovirus, retrovirus, AAV, vaccinia virus or herpes virus. These are representative examples only and macromolecular complexes within the scope of the present invention may include virtually any complex that may be attached to a targeting peptide and administered to a subject. In other preferred embodiments, the isolated peptide may be attached to a eukaryotic expression vector, more preferably a gene therapy vector.

In a further embodiment, the isolated peptide may be attached to a solid support, preferably magnetic beads, Sepharose beads, agarose beads, a nitrocellulose membrane, a nylon membrane, a column chromatography matrix, a high performance liquid chromatography (HPLC) matrix, a fast performance liquid chromatography (FPLC) matrix, a microtiter plate or a microchip.

In still further embodiments, the invention concerns protein fusion constructs comprising any one of the aforementioned LPR targeting peptides fused to a selected protein to form a protein fusion construct, preferably wherein the resultant protein fusion construct, by virtue of its further inclusion of the LPR targeting moiety, is a man-made and not a naturally occurring protein. Generally speaking, in such preferred embodiments, such protein fusion constructs can be prepared using any of the above-mentioned classes of molecules.

In still further embodiments, the invention concerns preparation of VEGFR-1/NRP-1 targeted construct comprising obtaining an LPR targeting peptide as described above and attaching the peptide to a molecule to prepare the construct, preferably by covalent attachment. As mentioned, where the molecule to be targeted is a protein or peptide, preferred targeting constructs will be those wherein the targeting peptide is attached at or near the amino or carboxy terminus of such a molecule.

The present invention is also directed to a method of targeting the delivery of a molecule or protein to cells that express VEGFR-1 or NRP-1, wherein the method includes obtaining an LPR targeting peptide or protein fusion construct as described above, or a targeted construct prepared as described above, and administering the peptide or protein fusion construct to a cell population, wherein the population includes cells that express VEGFR-1 or NRP-1, to thereby deliver the molecule or protein to said cells. Generally speaking, where the conjugate or fusion construct is intended for diagnostic or therapeutic application to a subject, such as a human subject, the conjugate or fusion construct is formulated in a pharmaceutically acceptable composition and the composition is administered to the subject.

It is contemplated that in the case of the therapeutic treatment of patients with a disease or disorder, the subject will typically be in need of anti-angiogenesis therapy. Such disease and disorders include hyperproliferative diseases, a weight disorder, obesity, diabetes, asthma, arthritis, cirrhosis or an ocular disease. Exemplary hyperproliferative diseases contemplated to be amenable to therapy using therapeutic conjugates in accordance with the invention, include rheumatoid arthritis, inflammatory bowel disease, osteoarthritis, leiomyomas, adenomas, lipomas, hemangiomas, fibromas, vascular occlusion, restenosis, atherosclerosis, pre-neoplastic lesions (such as adenomatous hyperplasia and prostatic intraepithelial neoplasia), carcinoma in situ, oral hairy leukoplakia, or psoriasis.

The invention also contemplates that the conjugates of the invention will be useful in the treatment of a wide range of cancers, particularly those cancers that are highly angiogenic. Exemplary cancers include cancers of the gum, tongue, lung, skin, liver, kidney, eye, brain, leukemia, mesothelioma, neuroblastoma, head, neck, breast, pancreatic, prostate, renal, bone, testicular, ovarian, mesothelioma, cervical, liver, cervical, head and neck, bone, esophageal, uterine, bladder, gastrointestinal, lymphoma, brain, colon, sarcoma, stomach, and bladder.

In still further embodiments, it is contemplated that the subject to be treated has an ocular disease or disorder characterized by intraocular cellular proliferation or neovascularization. Exemplary disorders include age-related macular degeneration, proliferative diabetic retinopathy, retinopathy of prematurity, glaucoma, proliferative vitreoretinopathy, neovascularization due to ocular ischemic syndrome, neovascularization due to branch retinal vein occlusion, neovascularization due to central retinal vein occlusion, or neovascularization due to sickle cell retinopathy.

In other embodiments, the invention contemplates that conjugates of the present invention will be useful in the treatment of weight disorders such as obesity.

### BRIEF DESCRIPTION OF THE DRAWINGS

FIGS. 1A, 1B. _{D}(LPR) inhibits neovascularization in vivo. Representative pictures of Matrigel plugs containing 500 μg/ml of _{D}(LPR) or control peptide after 7-days implant (a, lower panel). The Matrigel plugs were excised and angiogenesis was quantified by measuring the hemoglobin content in the Matrigel matrix. The bar graph shows representative animals from the same experiment (a, upper panel). (b) Number of vessels positive for human von Willebrand factor (P<0.01).

FIGS. 2A, 2B, 2C. Inhibition of ischemia-induced retinal angiogenesis by _{D}(LPR). (a) Retinal neovascularization was induced in C57B6 neonatal mice by exposure to 75% oxygen, followed by _{D}(LPR) treatment (daily injections at 20 mg/Kg). (b) H&E retina sections (day P19) showed a significant reduction in the formation of new blood vessels at the retinal inner surface (arrows) compared with the control animals. (c) Inner surface endothelial nuclei quantification at day P19.

FIGS. 3A, 3B. Treatment of tumor bearing mice with _{D}(LPR) reduces tumor growth. Balb/c mice bearing EF43.fgf4 derived tumors were divided in groups (N=7) and treated daily with 50 mg/Kg of _{D}(LPR) or its cyclic form _{D}(CLPR), control peptide or vehicle only. (a) After five days of treatment, animals receiving _{D}(LPR) or its cyclic form _{D}(CLPRC) showed reduced tumor volume compared to control animals. (b) Box plot shows the median and variance. The difference in tumor volume between the animals receiving _{D}(LPR) or its cyclic form _{D}(CLPRC) was statistically significant (P<0.02). Two independent experiments were performed with similar results.

FIGS. 4A, 4B, 4C. Obesity Treatment using VEGF-mimic compound _{D}(CLPRC). Obese mice (C57BL/6) fed with a high-calorie and high-fat diet (weight between 40 and 50 g) were used for this study. Animals were divided in four groups and treated daily with (a) VEGF-mimic peptide _{D}(CLPRC) injected intraperitoneally (50 mg/Kg); (b) Fat-zapper peptide (CKGGRAKDC-GG-_{D}(KLAKLAK)₂; Kolonin et al., 2004) injected subcutaneously at 1 mg/Kg in combination with the VEGF-mimic peptide _{D}(CLPRC) at 50 mg/Kg; or (c) fat-zapper at 3 mg/Kg. Animals in the control group were injected with vehicle only (phosphate saline buffered solution, PBS).

### DESCRIPTION OF ILLUSTRATIVE EMBODIMENTS

### 1. Overview

Peptides identified by combinatorial libraries are important leads toward drug discovery and design. They can be readily synthesized and easily modified with a variety of functional groups, providing science and medicine with powerful tools for rational drug design and selective targeting. Because of their smaller molecular weight compared with macromolecules like antibodies, peptides have an advantage in tissue permeability and biodistribution making them excellent lead compounds for drug discovery and development (reviewed by Falciani et al., 2005). In fact, peptides identified by phage display have been successfully used in vivo in targeted therapies to deliver chemotherapics (Arap, 1998 #10), pro-apoptotic peptides (Ellerby, 1999 #69), or to deliver viruses for imaging and gene-therapy (Hajitou, 2006 #6744). However, their applicability in drug development has been hampered by difficulties.

Peptides themselves are often not suitable drugs as they are quickly degraded by proteases and cleared from plasma. Protease expression is often up-regulated in biological process in which cell proliferation, migration and tissue remodeling are necessary (common to most pathological process such as angiogenesis), resulting in increased local proteolytic activity and peptide degradation. From the drug design perspective, peptides often display broad conformational variability making structural studies cumbersome and challenging (Giordano et al., 2005). Therefore, the design of peptidomimetic compounds based on peptide leads identified by phage display can be an arduous task, and is often limited to pharmaceutical companies or laboratories with synthesis capabilities and access to large chemical libraries.

Angiogenesis is the sprouting of new blood vessels from pre-existing ones and is an essential component in tumor growth and metastasis (Folkman, 1971) as well as several pathological disorders such as diabetes, psoriasis, obesity, and rheumatoid arthritis (Carmeliet, 2005). During adulthood angiogenesis occurs only during wound healing, pregnancy and the menstrual cycle, and therefore, drugs that target angiogenic blood vessels are likely to have important clinical applications in many diseases (Carmeliet et al., 2005). Vascular endothelial growth factor (VEGF) and their receptors have been the center of attention in the field due to their pivotal role in vessel development. VEGF exerts its effects by binding to tyrosine kinase receptors (VEGFR-1, VEGFR-2) and to neuropilin-1 (NRP-1) (Olsson et al., 2006). Most of the intracellular signaling and mitogenic effects of VEGF are mediated by VEGFR-2 and several drugs targeting this pathway are currently under investigation in the clinic (Cardones & Banez, 2006; Schneider & Sledge, 2007). Albeit being important players in the process, VEGFR-1 and NRP-1 initially failed to generate enough enthusiasm as potential therapeutic targets. This has changed and research generated in the past few years suggest that both receptors have a prominent role in angiogenesis (Luttun et al., 2004; Wu et al., 2006; Pan et al., 2007). Gene deletion studies have shown that VEGFR-1 and NPR-1 are essential during vessel development. Both molecules are receptors for VEGF and placental growth factor (P1GF), and the latter in conjunction with VEGFR-1 has been implicated in pathological angiogenesis (Carmeliet et al., 2001), tumor growth (Luttun et al., 2002), enhancing of the cellular signaling by VEGFR-1/VEGFR-2 cross-activation (Autiero et al., 2003) and recruitment of progenitor cells from the bone-marrow during neovascularization (Jin et al, 2006; Li et al., 2006). Recent reports have also suggested that the recruitment of VEGFR1+ haematopoietic progenitors is important for the initiation of tumor metastasis (Kaplan et al., 2005). Likewise, NRP-1 not only augments binding of VEGF to VEGFR-2 (Soker et al., 2002) but also induce endothelial cell attachment and migration independent of VEGFR-2 activation (Wang et al., 2003; Murga et al., 2005). Monoclonal antibodies directed against the VEGF binding domain of NRP-1 and against VEGFR-1, both, reduce angiogenesis reduce tumor growth (Wu et al., 2006; Pan et al., 2007). Therefore, drugs that target the VEGFR-1 and NRP-1 pathways are thus likely to find important applications in the clinic.

The present invention provides unique angiogenesis inhibitors and VEGFR-1 targeting agents, LPR and _{D}(LPR), which exhibit a significant reduction in angiogenesis in three different assays. _{D}(LPR) also inhibited neovascularization in two animal models after systemic administration. Given the resistance of _{D}(LPR) to degradation against the mixture of pancreatic enzymes, these data indicate that this compound, and larger peptide structures that incorporate this sequence, will apparently survive the digestive tract and could be administered orally to patients. Accordingly, the present invention overcomes deficiencies in the prior art by both identifying the LPR motif for the preparation of compositions to selectively target VEGFR-1/NRP-1, therapeutic and/or diagnostic agents, e.g., for the treatment and/or detection of neovascular or angiogenic VEGF associated disorders, including but not limited to cancer, obesity, diabetes, asthma, arthritis, cirrhosis and ocular diseases.

In certain embodiments, the invention concerns particular targeting moieties that one desires to target to VEGFR-1/NPR-1 expressing cells, including most generally peptides, polypeptides and proteins modified to include the LPR motif either internally or, more preferrably, at or near the N or C terminus of such a peptide or protein. Notably, while the D amino acid form is preferred due to its substantial resistance to the degrading effects of proteases, the present invention does not exclude the use of the less preferred L form, or mixtures of D and L amino acids. Particular embodiments concern VEGFR-1/NPR-1 targeting moieties that are operatively coupled to therapeutic or diagnostic agents. In certain embodiments, a therapeutic agent is a virus that can be engineered to express or has incorporated in or associated with its viral envelope or fiber proteins VEGFR-1/NPR-1 targeting peptides. Targeted viruses may then be used for gene therapy for the treatment of various disease states, including cancer. The ability to selectively target VEGFR-1/NPR-1 in the vasculature in and/or near tumors with peptides, modified peptides, antibodies, viruses, and/or other affinity reagents provides a significant advantage for the treatment of cancer that may result in an increased efficacy and potency.

### 2. Definitions

As used herein in the specification, “a” or “an” may mean one or more. As used herein in the claim(s), in conjunction with the word “comprising,” the words “a” or “an” may mean one or more than one. As used herein “another” may mean at least a second or more of an item.

A “targeting moiety” is a term that encompasses various types of affinity reagents that may be used to enhance the localization or binding of a substance to a particular location in an animal, including organs, tissues, particular cell types, diseased tissues or tumors. Targeting moieties may include peptides, peptide mimetics, polypeptides, antibodies, antibody-like molecules, nucleic acids, aptamers, and fragments thereof. Targeting moieties also include small molecules. In certain embodiments, a targeting moiety will enhance the localization of a substance to cells expressing VEGFR-1/NRP-1 extracellularly, i.e., VEGFR-1/NRP-1 being associated with the cell surface or associated with surrounding extracelluar matrix. Selective binding of a targeting moiety of the present invention, e.g., a targeting peptide, as well as variants and fragments thereof is when the targeting moiety binds a target (e.g., VEGFR-1/NRP-1) and does not significantly bind to unrelated proteins. A targeting moiety is still considered to selectively bind even if it also binds to other proteins that are not substantially homologous with the target so long as such proteins share homology with a fragment or domain of the peptide target of the antibody. In this case, it would be understood that target moiety binding to the target is still selective despite some degree of cross-reactivity. Typically, the degree of cross-reactivity can be determined and differentiated from binding to the target.

A “targeting peptide” is a peptide comprising a contiguous sequence of LPR amino acids, which is characterized by selective localization to an organ, tissue or cell type, which includes specific binding with an extracellar protein or molecule that is specifically expressed or produced in a specific tissue or cell type(s). Selective localization may be determined, for example, by methods disclosed below, wherein the putative targeting peptide sequence is incorporated into a protein that is displayed on the outer surface of a phage.

A “subject” refers generally to a mammal. In certain embodiments, the subject, is a mouse, rabbit, a pig, a horse, a cow, a cat, a dog, a sheep, a goat, or a primate. In specific embodiments, the subject is a human.

### 3. Proteins and Peptides

In certain embodiments, the present invention concerns novel compositions comprising at least one protein or peptide. As used herein, a protein or peptide generally refers, but is not limited to, a protein of greater than about 200 amino acids, up to a full length sequence translated from a gene; a polypeptide of greater than about 100 amino acids; and/or a peptide of from about 3 to about 100 amino acids. For convenience, the terms “protein,” “polypeptide” and “peptide” are used interchangeably herein.

In certain embodiments the size of at least one protein or peptide may comprise, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino acid residues, or any range of amino acid residues derivable therein (e.g., about 200 to about 2500 amino acid residues).

As used herein, an “amino acid residue” refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moiety. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid, including but not limited to Aad, 2-Aminoadipic acid; EtAsn, N-Ethylasparagine; Baad, 3-Aminoadipic acid, Hyl, Hydroxylysine; Bala, β-alanine, β-Amino-propionic acid; AHyl, allo-Hydroxylysine; Abu, 2-Aminobutyric acid; 3Hyp, 3-Hydroxyproline; 4Abu, 4-Aminobutyric acid, piperidinic acid; 4Hyp, 4-Hydroxyproline; Acp, 6-Aminocaproic acid, Ide, Isodesmosine; Ahe, 2-Aminoheptanoic acid; AIle, allo-Isoleucine; Aib, 2-Aminoisobutyric acid; MeGly, N-Methylglycine, sarcosine; Baib, 3-Aminoisobutyric acid; MeIle, N-Methylisoleucine; Apm, 2-Aminopimelic acid; MeLys, 6-N-Methyllysine; Dbu, 2,4-Diaminobutyric acid; MeVal, N-Methylvaline; Des, Desmosine; Nva, Norvaline; Dpm, 2,2′-Diaminopimelic acid; Nle, Norleucine; Dpr, 2,3-Diaminopropionic acid; Om, Ornithine; and EtGly, N-Ethylglycine.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases (world wide web at ncbi.nlm.nih.gov). The coding regions for known genes may be amplified and/or expressed using the techniques disclosed herein or as would be know to those of ordinary skill in the art. Alternatively, various commercial preparations of proteins, polypeptides and peptides are known to those of skill in the art.

### 4. Fusion Proteins

Other embodiments of protein conjugates concern fusion proteins. These molecules generally have all or a substantial portion of a targeting peptide (e.g., an LPR targeting peptide), linked at the N- or C-terminus, to all or a portion of a second polypeptide or protein. For example, fusions may employ leader sequences from other species to permit the recombinant expression of a protein in a heterologous host. Another useful fusion includes the addition of an immunologically active domain, such as an antibody epitope, to, for example, facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. In preferred embodiments, the fusion proteins of the instant invention comprise an LPR targeting peptide linked to a therapeutic protein or peptide. Examples of proteins or peptides that may be incorporated into a fusion protein include cytostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins. These examples are not meant to be limiting and it is contemplated that within the scope of the present invention virtually any protein or peptide could be incorporated into a fusion protein comprising a targeting peptide. Methods of generating fusion proteins are well known to those of skill in the art. Such proteins can be produced, for example, by chemical attachment using bifunctional cross-linking reagents, by de novo synthesis of the complete fusion protein, or by attachment of a DNA sequence encoding the targeting peptide to a DNA sequence encoding the second peptide or protein, followed by expression of the intact fusion protein.

### 5. Protein Purification

In certain embodiments a protein or peptide may be isolated or purified. Protein purification techniques are well known to those of skill in the art. These techniques involve, at one level, the homogenization and crude fractionation of the cells, tissue or organ to polypeptide and non-polypeptide fractions. The protein or polypeptide of interest may be further purified using chromatographic and electrophoretic techniques to achieve partial or complete purification (or purification to homogeneity). Analytical methods particularly suited to the preparation of a pure peptide are ion-exchange chromatography, gel exclusion chromatography, polyacrylamide gel electrophoresis, affinity chromatography, immunoaffinity chromatography and isoelectric focusing. An example of receptor protein purification by affinity chromatography is disclosed in U.S. Pat. No. 5,206,347, the entire text of which is incorporated herein by reference. A particularly efficient method of purifying peptides is fast performance liquid chromatography (FPLC) or even high performance liquid chromatography (HPLC).

A purified protein or peptide is intended to refer to a composition, isolatable from other components, wherein the protein or peptide is purified to any degree relative to its naturally-obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various methods for quantifying the degree of purification of the protein or peptide are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “-fold purification number.” The actual units used to represent the amount of activity will, of course, be dependent upon the particular assay technique chosen to follow the purification, and whether or not the expressed protein or peptide exhibits a detectable activity.

Various techniques suitable for use in protein purification are well known to those of skill in the art. These include, for example, precipitation with ammonium sulphate, PEG, antibodies and the like, or by heat denaturation, followed by: centrifugation; chromatography steps such as ion exchange, gel filtration, reverse phase, hydroxylapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance to be isolated and a molecule to which it can specifically bind. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.). The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand.

### 6. Synthetic Peptides

Because of their relatively small size, the targeting peptides of the invention can be synthesized in solution or on a solid support in accordance with conventional techniques. Various automatic synthesizers are commercially available and can be used in accordance with known protocols. See, for example, Stewart and Young, 1984; Tam et al., 1983; Merrifield, 1986; Barany and Merrifield, 1979, each incorporated herein by reference. Short peptide sequences, usually from about 6 up to about 35 to 50 amino acids, can be readily synthesized by such methods. Alternatively, recombinant DNA technology may be employed wherein a nucleotide sequence which encodes a peptide of the invention is inserted into an expression vector, transformed or transfected into an appropriate host cell, and cultivated under conditions suitable for expression.

### 7. Therapeutic or Diagnostic Conjugates

Targeting moieties identified using these methods may be coupled or attached to various substances, including therapeutic or diagnostic agents, for the selective delivery of the conjugate to a desired organ, tissue or cell type in the mouse model system. For example, targeted delivery of chemotherapeutic agents and proapoptotic peptides to receptors located in tumor angiogenic vasculature result in a marked increase in therapeutic efficacy and a decrease in systemic toxicity in tumor bearing mouse models (Arap et al., 1998; Ellerby et al., 1999).

Embodiments of the invention are directed to the treatment of neovascularization associated with various disease states, such as tumor vasculature. In addition to tumor growth, angiogenesis is important in other diseases. Uncontrolled angiogenesis contributes to the progression of rheumatoid arthritis, diabetic retinopathy, endometriosis, age-related macular degeneration, and psoriasis. Growth of blood vessels results in the formation of hemangiomas and arteriovenous malformations that cause a variety of clinical problems ranging from cosmetic complications to life-threatening hemorrhages. Further embodiments of the invention are directed to treatment of these exemplary disease states as well as other associated with neo-vascularization.

Alternatively, the upregulation of VEGFR-1/NRP-1 or the targeting of angiogensis promoting compounds or substances may be used to promote angiogenesis. Upregulation of VEGFR-1/NRP-1 may be accomplished by delivery of an VEGFR-1 (i.e., Flt-1) or NRP-1 transgene, which in turn may be delivered by various gene therapy vectors known to those of skill in the art.

A. Cytokines and Chemokines

In certain embodiments, it may be desirable to couple specific bioactive agents to one or more targeting moieties for targeted delivery to an organ, tissue or cell type. Such agents include, but are not limited to, cytokines, chemokines, pro-apoptosis factors and anti-angiogenic factors. The term “cytokine” is a generic term for proteins released by one cell population that act on another cell as intercellular mediators.

Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, LIF, G-CSF, GM-CSF, M-CSF, EPO, kit-ligand or FLT-3, angiostatin, thrombospondin, endostatin, tumor necrosis factor, and LT. As used herein, the term “cytokine” includes proteins from natural sources or from recombinant cell culture and biologically active equivalents of the native sequence cytokines.

Chemokines generally act as chemoattractants to recruit immune effector cells to the site of chemokine expression. It may be advantageous to express a particular chemokine gene in combination with, for example, a cytokine gene, to enhance the recruitment of other immune system components to the site of treatment. Chemokines include, but are not limited to, RANTES, MCAF, MIP1-alpha, MIP1-Beta, and IP-10. The skilled artisan will recognize that certain cytokines are also known to have chemoattractant effects and could also be classified under the term chemokines.

B. Imaging Agents and Radioisotopes

In certain embodiments, the targeting moieties of the present invention may be attached to imaging agents of use for imaging and diagnosis of various diseased organs, tissues or cell types. Many appropriate imaging agents are known in the art, as are methods for their attachment to proteins or peptides (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the protein or peptide (U.S. Pat. No. 4,472,509). Proteins or peptides also may be reacted with an enzyme in the presence of a coupling agent such as glutaraldehyde or periodate. Conjugates with fluorescein markers are prepared in the presence of these coupling agents or by reaction with an isothiocyanate.

Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (1H), gold (III), lead (H), and especially bismuth (III).

Radioisotopes of potential use as imaging or therapeutic agents include ²¹¹astatine, ¹⁴-carbon, ⁵¹ chromium, ³⁶-chlorine, ⁵⁷cobalt, ⁵⁸cobalt, ⁶⁷copper, ¹⁵²Eu, ⁶⁷gallium, ³hydrogen, ¹²³iodine, ¹²⁵iodine, ¹²⁵iodine, ¹¹¹indium, ⁵⁹iron, ³²phosphorus, ¹⁸⁶rhenium, ¹⁸⁸rhenium, ⁷⁵selenium, ³⁵sulphur, ^{99m}technicium and ⁹⁰yttrium. ¹²⁵I is often being preferred for use in certain embodiments, and ^{99m}technicium and ¹¹¹indium are also often preferred due to their low energy and suitability for long range detection.

Radioactively labeled proteins or peptides of the present invention may be produced according to well-known methods in the art. For instance, they can be iodinated by contact with sodium or potassium iodide and a chemical oxidizing agent such as sodium hypochlorite, or an enzymatic oxidizing agent, such as lactoperoxidase. Proteins or peptides according to the invention may be labeled with ^{99m}technetium by ligand exchange process, for example, by reducing pertechnate with stannous solution, chelating the reduced technetium onto a Sephadex column and applying the peptide to this column or by direct labeling techniques, e.g., by incubating pertechnate, a reducing agent such as SNCl₂, a buffer solution such as sodium-potassium phthalate solution, and the peptide. Intermediary functional groups that are often used to bind radioisotopes that exist as metallic ions to peptides are diethylenetriaminepenta-acetic acid (DTPA) and ethylene diaminetetra-acetic acid (EDTA). Also contemplated for use are fluorescent labels, including rhodamine, fluorescein isothiocyanate and renographin.

In certain embodiments, the claimed proteins or peptides may be linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

In still further embodiments, a targeting moiety may be operatively coupled to a nanoparticle. Nanoparticles include, but are not limited to colloidal gold and silver nanoparticles. Metal nanoparticles exhibit colors in the visible spectral region. It is believed that these colors are the result of excitation of surface plasmon resonances in the metal particles and are extremely sensitive to size, shape, and aggregation state of particles; dielectric properties of the surrounding medium; adsorption of ions on the surface of the particles (see, e.g., U.S. Patent Application 20040023415, which is incorporated herein by reference).

C. Cross-Linkers

Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. Nos. 5,603,872 and 5,401,511, each specifically incorporated herein by reference in its entirety. Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites are dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the complex chemistry of cross-linking, linkage of the amine residues of the recognizing substance and liposomes is established.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups.

### 8. Nucleic Acids

Nucleic acids according to the present invention may encode a targeting peptide, a targeting antibody, a targeting antibody fragment, a therapeutic polypeptide, a fusion protein or other protein or peptide. The nucleic acid may be derived from genomic DNA, complementary DNA (cDNA) or synthetic DNA.

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of almost any size, determined in part by the length of the encoded protein or peptide.

It is contemplated that targeting peptides and fusion proteins may be encoded by any nucleic acid sequence that encodes the appropriate amino acid sequence. The design and production of nucleic acids encoding a desired amino acid sequence is well known to those of skill in the art, using standardized codon tables. In preferred embodiments, the codons selected for encoding each amino acid may be modified to optimize expression of the nucleic acid in the host cell of interest.

### 9. Targeted Delivery of Gene Therapy Vectors

There are a number of ways in which gene therapy vectors may be introduced into cells. In certain embodiments of the invention, the gene therapy vector comprises a virus. The ability of certain viruses to enter cells via receptor-mediated endocytosis, to integrate into host cell genome or be maintained episomally, and express viral genes stably and efficiently have made them attractive candidates for the transfer of foreign genes into mammalian cells (Ridgeway, 1988; Nicolas and Rubinstein, 1988.; Baichwal and Sugden, 1986; Temin, 1986). Preferred gene therapy vectors are generally viral vectors. DNA viruses used as gene therapy vectors include the papovaviruses (e.g., simian virus 40, bovine papilloma virus, and polyoma) (Ridgeway, 1988; Baichwal and Sugden, 1986) and adenoviruses (Ridgeway, 1988; Baichwal and Sugden, 1986).

One of the preferred methods for in vivo delivery involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include, but is not limited to, constructs containing adenovirus sequences sufficient to (a) support packaging of the construct and (b) to express an antisense or a sense polynucleotide that has been cloned therein.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic innoculation into the brain (Le Gal La Salle et al., 1993).

In preferred embodiments, certain advantages may be gained from coupling therapeutic molecules or substances to LPR targeting moieties that target the vasculature of diseased tissues, e.g., tumors or neo-vascular beds. Specifically, moieties that home to tumor vasculature have been coupled to cytotoxic drugs or proapoptotic peptides to yield compounds were more effective and less toxic than the parental compounds in experimental models of mice bearing tumor xenografts (Arap et al., 1998; Ellerby et al, 1999). The insertion of the RGD-4C peptide into a surface protein of an adenovirus has produced an adenoviral vector that may be used for tumor targeted gene therapy (Arap et al., 1998).

A “fiber protein” according to the invention preferably comprises an adenoviral fiber protein. Any one of the serotypes of human or nonhuman adenovirus (e.g., a chimeric fiber protein) can be used as the source of the fiber protein or fiber gene. Optimally, however, the adenovirus is an Ad2 or Ad5 adenovirus. (see, U.S. Pat. No. 6,649,407, which is incorporated herein by refernce in its entirety).

The fiber protein is “chimeric” in that it comprises amino acid residues that are not typically found in the protein as isolated from wild-type adenovirus (i.e., comprising the native protein, or wild-type protein). The fiber protein thus comprises a “normative amino acid sequence”. “Normative amino acid sequence” means a sequence of any suitable length, preferably from about 3 to about 200 amino acids, optimally from about 3 to about 30 amino acids. Desirably, the normative amino acid sequence is introduced into the fiber protein at the level of gene expression (i.e., by introduction of a “nucleic acid sequence that encodes a normative amino acid sequence”). Such a normative amino acid sequence either is introduced in place of adenoviral sequences, or in addition to adenoviral sequences. Regardless of the nature of the introduction, its integration into an adenoviral fiber protein at the level of either DNA or protein, results in the generation of a peptide motif (i.e., a peptide binding motif) in the resultant chimeric fiber protein.

The peptide motif allows for cell targeting, for instance, by comprising a targeting moiety of the invention, and/or a ligand for a cell surface binding site. The peptide motif optionally can comprise other elements of use in cell targeting (e.g., a single-chain antibody sequence). The peptide binding motif may be generated by the insertion, and may comprise, for instance, native and normative sequences, or may be entirely made up of normative sequences. The peptide motif that results from the insertion of the normative amino acid sequence into the chimeric fiber protein can be either a high affinity peptide (i.e., one that binds its cognate binding site, e.g., VEGFR-1/NRP-1, when provided at a relatively low concentration) or a low affinity peptide (i.e., one that binds its cognate binding site, e.g., VEGFR-1/NRP-1, when provided at a relatively high concentration). Preferably, however, the resultant peptide motif is a high affinity motif, particularly one that has a high affinity for its cognate binding site due to its constraint within the adenovirus fiber protein.

Other gene transfer vectors may be constructed from retroviruses. (Coffin, 1990.) In order to construct a retroviral vector, a nucleic acid encoding protein of interest is inserted into the viral genome in the place of certain viral sequences to produce a virus that is replication-defective. In order to produce virions, a packaging cell line containing the gag, pol, and env genes, but without the LTR and packaging components, is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are capable of infecting a broad variety of cell types. However, integration and stable expression require the division of host cells (Paskind et al., 1975).

Other viral vectors may be employed as targeted gene therapy vectors. Vectors derived from viruses such as vaccinia virus (Ridgeway, 1988; Baichwal and Sugden, 1986), adeno-associated virus (AAV) (Ridgeway, 1988; Baichwal and Sugden, 1986; Hermonat and Muzycska, 1984), and herpes viruses may be employed.

In a further embodiment of the invention, gene therapy construct may be entrapped in a liposome. Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa, and hepatoma cells. Nicolau et al., (1987.) accomplished successful liposome-mediated gene transfer in rats after intravenous injection.

Gene therapy vectors of the invention may comprise various transgenes, which are typically encoded DNA or RNA of an expression vector. Gene therapy may be used for the expression of a therapeutic gene, expression of VEGFR-1/NRP-1 to enhance neo-vascularization or for the inhibition of VEGFR-1/NRP-1 expression for the treatment of disease states associated with neo-vascularization. DNA may be in form of cDNA, in vitro polymerized DNA, plasmid DNA, parts of a plasmid DNA, genetic material derived from a virus, linear DNA, vectors (P1, PAC, BAC, YAC, artificial chromosomes), expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), ribozymes, or derivatives of these groups. An anti-sense polynucleotide is a polynucleotide that interferes with the function of DNA and/or RNA. Antisense polynucleotides include, but are not limited to: morpholinos, 2′-O-methyl polynucleotides, DNA, RNA and the like. SiRNA comprises a double stranded structure typically containing 15-50 base pairs and preferably 21-25 base pairs and having a nucleotide sequence identical or nearly identical to an expressed target gene or RNA within the cell. Interference may result in suppression of expression. In addition, DNA and RNA may be single, double, triple, or quadruple stranded.

### 10. Pharmaceutical Compositions

Pharmaceutical compositions of the present invention comprise an effective amount of one or more compositions including a targeting moiety as described herein dissolved or dispersed in a pharmaceutically acceptable carrier. The phrases “pharmaceutical” or “pharmacologically acceptable” refer to molecular entities and compositions that do not produce an adverse, allergic or other untoward reaction when administered to an animal, such as, for example, a human, as appropriate. The preparation of a pharmaceutical composition that contains at least one composition of the present invention (e.g., LPR targeting moieties) or an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed., 1990, incorporated herein by reference). Except insofar as any conventional carrier is incompatible with the active ingredient, its use in the therapeutic or pharmaceutical compositions is contemplated.

The therapeutic and diagnostic compositions of the present invention may comprise different types of carriers depending on whether it is to be administered in solid, liquid or aerosol form, and whether it need to be sterile for such routes of administration. It is contemplated that compositions of the present invention can be administered by any method known to those of ordinary skill in the art, such as orally, intravenously, intradermally, intraarterially, intrathecally, intraocularly, subconjunctivally, subretinally, intravitreally, into the anterior chamber of the eye, into the sub-Tenon's space of the eye, topically, intraperitoneally, intralesionally, intracranially, intraarticularly, intrapleurally, intratracheally, intratumorally, intramuscularly, intraperitoneally, subcutaneously, intravesicularlly, inhalation (e.g., aerosol inhalation), injection, infusion, continuous infusion, localized perfusion bathing target cells directly, via a catheter, via a lavage, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, about 1 mg peptide/kg body weight, about 5 mg/kg body weight, about 10 mg/kg body weight, about 50 mg/kg/body weight, about 100 mg/kg body weight, about 200 mg/kg body weight or more per administration, and any range derivable therein. In non-limiting examples of a derivable range from the numbers listed herein, a range of about 1 mg/kg body weight to about 100 mg/kg body weight is preferred, with between 20 and 50 mg/kg being particularly preferred, in multiple daily doses (similar to ibuprofin, aspirin, etc., every 4-8 h).

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In embodiments where the composition is in a liquid form, a carrier can be a solvent or dispersion medium comprising but not limited to, water, ethanol, polyol (e.g., glycerol, propylene glycol, liquid polyethylene glycol), lipids (e.g., triglycerides, vegetable oils, liposomes) and combinations thereof. In many cases, it will be preferable to include isotonic agents, such as, for example, sugars, sodium chloride or combinations thereof.

Sterile injectable solutions are prepared by incorporating the LPR targeting moiety or conjugate thereof in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

The compositions set forth herein may optionally include on or more secondary therapeutic agents directed to treatment or prevention of any of the diseases set forth herein.

### 11. Therapeutic Agents

In certain embodiments, therapeutic agents may be operatively coupled to a targeting peptide or fusion protein for selective delivery to, for example, tumor vasculature expressing VEGFR-1/NRP-1. Agents or factors suitable for use may include any chemical compound that induces apoptosis, cell death, cell stasis and/or anti-angiogenesis.

A. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins that share in common structural and sequence homologies. These different family members have been shown to either possess similar functions to Bcl-2 (e.g., Bcl_{XL}, Bcl_{W}, Bcl_{S}, Mcl-1, Al, Bfl-1) or counteract Bcl-2 function and promote cell death (e.g., Bax, Bak, Bik, Bim, Bid, Bad, Harakiri).

B. Angiogenic Inhibitors

In certain embodiments the present invention may concern administration of targeting moieties operatively coupled to anti-angiogenic agents, such as angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4 or minocycline.

Proliferation of tumors cells relies heavily on extensive tumor vascularization, which accompanies cancer progression. Thus, inhibition of new blood vessel formation with anti-angiogenic agents and targeted destruction of existing blood vessels have been introduced as an effective and relatively non-toxic approach to tumor treatment. (Arap et al., 1998; Arap et al., 1998; Ellerby et al., 1999). A variety of anti-angiogenic agents and/or blood vessel inhibitors are known. (e.g., Folkman, 1997; Eliceiri and Cheresh, 2001).

C. Cytotoxic Agents

Chemotherapeutic (cytotoxic) agents may be used to treat various disease states, including cancer. Chemotherapeutic (cytotoxic) agents of potential use include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. Most chemotherapeutic agents fall into the categories of alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics” and in “Remington's Pharmaceutical Sciences” 15^{th} ed., pp 1035-1038 and 1570-1580, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Of course, all dosages and agents described herein are exemplary rather than limiting, and other doses or agents may be used by a skilled artisan for a specific patient or application. Any dosage in-between these points, or range derivable therein is also expected to be of use in the invention.

D. Alkylating Agents

Alkylating agents are drugs that directly interact with genomic DNA to prevent cells from proliferating. This category of chemotherapeutic drugs represents agents that affect all phases of the cell cycle, that is, they are not phase-specific. An alkylating agent, may include, but is not limited to, a nitrogen mustard, an ethylenimene, a methylmelamine, an alkyl sulfonate, a nitrosourea or a triazines. They include but are not limited to: busulfan, chlorambucil, cisplatin, cyclophosphamide (cytoxan), dacarbazine, ifosfamide, mechlorethamine (mustargen), and melphalan.

E. Antimetabolites

Antimetabolites disrupt DNA and RNA synthesis. Unlike alkylating agents, they specifically influence the cell cycle during S phase. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

F. Natural Products

Natural products generally refer to compounds originally isolated from a natural source, and identified as having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers.

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine.

Taxoids are a class of related compounds isolated from the bark of the ash tree, Taxus brevifolia. Taxoids include but are not limited to compounds such as docetaxel and paclitaxel. Paclitaxel binds to tubulin (at a site distinct from that used by the vinca alkaloids) and promotes the assembly of microtubules.

Vinca alkaloids are a type of plant alkaloid identified to have pharmaceutical activity. They include such compounds as vinblastine (VLB) and vincristine.

G. Antibiotics

Certain antibiotics have both antimicrobial and cytotoxic activity. These drugs also interfere with DNA by chemically inhibiting enzymes and mitosis or altering cellular membranes. These agents are not phase specific so they work in all phases of the cell cycle. Examples of cytotoxic antibiotics include, but are not limited to, bleomycin, dactinomycin, daunorubicin, doxorubicin (Adriamycin), plicamycin (mithramycin) and idarubicin.

H. Miscellaneous Agents

Miscellaneous cytotoxic agents that do not fall into the previous categories include, but are not limited to, platinum coordination complexes, anthracenediones, substituted ureas, methyl hydrazine derivatives, amsacrine, L-asparaginase, and tretinoin. Platinum coordination complexes include such compounds as carboplatin and cisplatin (cis-DDP). An exemplary anthracenedione is mitoxantrone. An exemplary substituted urea is hydroxyurea. An exemplary methyl hydrazine derivative is procarbazine (N-methylhydrazine, MIH). These examples are not limiting and it is contemplated that any known cytotoxic, cytostatic or cytocidal agent may be attached to targeting peptides and administered to a targeted organ, tissue or cell type within the scope of the invention.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Anti-Angiogenic Compound that Targets VEGF Pathways

### 1. Materials and Methods

Reagents and peptides. Peptides were synthesized and HPLC purified to our specifications with purity greater then 95%: L-Arg-L-Pro-L-Leu (RPL), D-Leu-D-Pro-D-Arg [_{D}(LPR)], D-Cys-D-Ala-D-Pro-D-Ala-D-Cys [_{D}(CAPAC); SEQ ID NO:6] by Polypeptide Laboratories (Torrence, Calif.) and D-Ala-D-Pro-D-Ala [_{D}(APA)] by Genemed Synthesis Inc. (San Francisco, Calif.). Recombinant receptors (VEGFR-1 and NPR-1) and growth factors (human VEGF₁₆₅) were obtained from R&D Systems (Minneapolis, Minn.). Heparin, Drabkin reagent, human hemoglobin, brij-35 were obtained from (Sigma-Aldrich, St. Louis, Mo.).

Animals. Mouse experiments were approved by the Animal Care and Use Committee of the University of Texas M. D. Anderson Cancer Center. C57BL/6 and Balb/c mice were commercially obtained (Harlan, Indianapolis). This study adhered to the Association for Research in Vision and Ophthalmology Statement for the Use of Animals in Ophthalmic and Vision Research.

Phage assay. Phage was prepared by infection of log-phase culture of E. coli K91kan, and overnight growth in Luria-Bertani (LB) media supplemented with kanamycin (100 μg/ml) and tetracyclin (20 μg/ml) at 37° C. and 250 rpm. Phage was precipiated from the media supernatant by the PEG/NaCl method, and phage titer determined by serial dilution and colony counting (Giordano, 2001). For the phage binding and competition assays, VEGFR-1, NRP-1 or BSA (10 μg/ml in PBS) was immobilized on microtiter wells overnight at 4° C. Wells were washed twice, blocked with PBS 3% BSA for 2 h at room temperature and incubated with 10⁹ TU of CPQPRPLC or negative control insertless (Fd-tet) phage in PBS 3% BSA. After 1 h at room temperature, wells were washed 10 times with PBS and phage bound to the immobilized receptors recovered by bacterial infection (Giordano, 2001).

Protease resistance assay. The _{D}(LPR) and RPL peptides were diluted to 500 μg/ml in PBS and incubated with increasing concentrations of pancreatin (Sigma-Aldrich, St. Louis, Mo.) for 2 h at 37° C. Samples were then analyzed by mass spectroscopy (MALDI-TOF).

Angiogenesis assays. The inventors used the in vivo matrigel angiogenesis assay in which growth factor reduced Matrigel matrix (BD Biosciences, Bedford, Mass.) impregnated with recombinant human VEGF₁₆₅ (1 μg/ml) and heparin (10 U/ml), containing or not the peptidomimetic compounds (500 μg), were implanted in vivo subcutaneous (0.5 ml) into the dorsal area of Balb-c mice. _{D}(CAPAC) was used as control peptidomimetic. After 7 days, mice were sacrificed, matrigel plugs were dissected out, photographed, homogenized in Brij 0.35% solution with the help of a Dounce homogenizer, and centrifuged for 5 min at 13.000 g. The supernatant was used in duplicate to measure hemoglobin (Hb) with Drabkin's reagent and the concentration of Hb calculated based on Hb standard measured simultaneously.

SCID mouse model of human angiogenesis. Mice were implanted with 10⁶ human dermal microendothelial cells (HDMEC) in Matrigel/scaffold, 2 scaffolds per mouse, one on each flank. Day 12 after implantation mice were treated daily with _{D}(LPR) or control peptidomimetic _{D}(APA) (100 μL intraperitoneal injections of a 2 mg/ml solution). Drugs were both formulated by dissolution in DMSO to a stock concentration of 20 mg/ml and fresh dilutions (1/10) in PBS made for each injection. Scaffolds were harvested and fixed in 10% formalin/PBS. Tissue sections were labeled with von Willebrand factor antibody (Neomarkers, Freemont, Calif.), visualized with AEC (DABCO) and counterstained with haematoxylin. Counts were performed under light microscope, six fields per scaffold at 200× magnification (n=6 for _{D}(LPR) and n=4 for control peptidomimetic). Statistical analysis was performed on Sigmastat.

Retinal neovascularization angiogenesis assay. For the retinal neovascularization assay the inventors used C57BL/6 mouse pups with their nursing mothers. Mice (P7) were exposed to 75% oxygen for 5 days, returned to room air (20.8% O₂)(P12) and injected daily with _{D}(LPR), control peptidomimetic _{D}(CAPAC; SEQ ID NO:6) (20 mg/Kg in phosphate buffered saline as vehicle) or vehicle for seven days (P12 to P18). For histological analysis, mice were sacrificed at the angiogenesis peak on P19, eyes were enucleated, fixed, serially sectioned, and stained with hematoxylin and eosin (H&E). Endothelial cell nuclei on the vitreous side of the internal limiting membrane were counted. At least 10 H&E-stained sections were evaluated per eye, and the average number of nuclei was counted from 4-6 eyes for each condition.

Tumor growth. The EF43.fgf4 cells were cultured in Dulbelco's modified Eegles medium supplemented with fetal bovine serum, glutamine and antibiotics. Cells were harvested before reaching confluence and injected subcutaneously in the mammary fatpad of Balb/c mice. Mier 10 days, tumor reached ˜50-80 mm³ and were then separated into four groups with seven animals (N=7). Animals in each group were treated with either vehicle only (phosphate saline buffered solution) or with control peptide _{D}(CAPAC) (SEQ ID NO:6) or _{D}(LPR) (at 50 mg/Kg) or with the cyclic version _{D}(CLPRC) (SEQ ID NO:7) (at 25 mg/Kg). Tumor volume was calculated by measuring the length of the long (L) and the short (S) sides of each tumor (V=S²×L×0.04).

Statistical analysis. For the in vivo experiments, statistical significance of the difference was computed by Kruskal-Wallis test (non parametric one factor ANOVA method) with p<0.05 for each treatment day. Wilcoxon Rank Sum test was used to further compute difference between each pair wise study groups on treatment day that showed statistical significance from Kruskal-Wallis test. Multiple comparisons were adjusted by Bejamini & Hochberg method. All the statistical analysis was computed using The R (Version 2.4.1) Project for Statistical Computing.

### 2. Experimental Results

To access whether the peptidomimetic compound _{D}(LPR) exhibited VEGFR-1 and NRP-1 binding competition experiments were performed. Binding of the parental CPQPRPLC (SEQ ID NO:8) phage to the immobilized VEGF receptors NRP-1 and VEGFR-1 was performed in the presence of increasing concentrations of RPL peptide or the _{D}(LPR) peptidomimetic of the present invention. As expected from our previous work (Giordano et al., 2005), the RPL peptide completely abrogated the binding of CPQPRPLC (SEQ ID NO:8) phage to both receptors. Interestingly, the _{D}(LPR) peptidomimetic also inhibited phage binding at very similar levels compared to RPL. Both peptides, RPL and _{D}(LPR), inhibited phage binding in a dose-dependent manner while a control peptide used at the highest concentration (100 μM) had no effect on CPQPRPLC (SEQ ID NO:8) phage binding. Because the concentration of peptide required to inhibit 50% of phage binding (IC₅₀) is inversely proportional to the affinity of the peptide for the receptor, our data suggests that both RPL and _{D}(LPR) have higher affinity for VEGFR-1 and NRP-1 compared to the CPQPRPLC (SEQ ID NO:8) peptide (Giordano et al., 2001). The IC₅₀ of _{D}(LPR) for both receptors is significantly lower (1-10 pM) compared to the IC₅₀ of CPQPRPLC (SEQ ID NO:8) previously described for VEGFR-1 (˜1 nM) or NRP-1 (˜50-100 nM) (Giordano et al., 2001).

Next, the resistance of _{D}(LPR) to proteolytic degradation was tested. Both, RPL and _{D}(LPR) were incubated with increasing concentrations of pancreatin (a mixture of several digestive enzymes produced by the pancreas), and the degradation products analyzed by mass spectroscopy. No breakdown products were observed with the _{D}(LPR) peptidomimetic at the highest ratio of enzyme per peptide concentration tested (400 pg/nmol). However, RPL peptide showed marked degradation with the presence of the PL dipeptide breakdown products. In summary, these data demonstrate that _{D}(LPR) is an RPL mimic that is less prone to proteolytic degradation, it binds with high affinity to VEGFR-1 and NRP-1 and it is a better drug-lead candidate to study the effects of the RPL motif in angiogenesis.

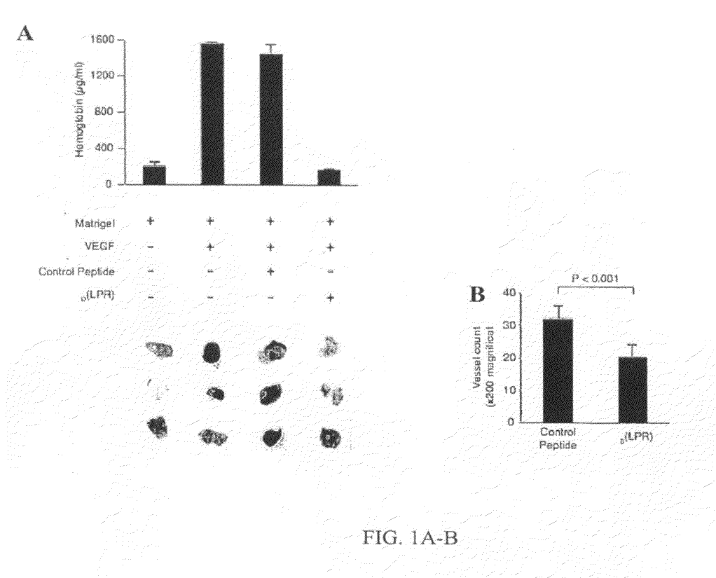

Having identified and characterized the _{D}(LPR) peptidomimetic compound, the effect of _{D}(LPR) on neo-vessel formation was investigated. For this purpose, two animal models of angiogenesis were employed: the in vivo Matrigel and the growth of human endothelial cells in a murine host (Nor et al., 2001). Initial studies were performed with the in vivo Matrigel assay model in which mice were injected subcutaneously with Matrigel containing VEGF₁₆₅ and the _{D}(LPR) peptidomimetic. After 7 days implantation, Matrigel plugs that had been impregnated with _{D}(LPR) showed diminished vascularization compared to the positive control Matrigel plugs with VEGF₁₆₅ only; no effect on neovascularization was observed in the plugs containing a control peptidomimetic (FIG. 1A).

Next, the effect of _{D}(LPR) in the SCID mouse model of human angiogenesis was investigated. In this model, human endothelial cells are cultured in vivo within polymer implants and grow to form microvessels, which then merge with the mouse capillaries. The neovessels are functional, lined up with human endothelial cells expressing angiogenesis markers, and transport murine blood cells. To assess the effect of _{D}(LPR) in this angiogenesis animal model, mice were implanted with scaffolds containing human endothelial cells and allowed to grow for 12 days. During this 12-day period, the endothelial cells formed nonfunctional tubular structures containing empty lumens, which slowly matured to fully function vessels containing murine blood cells (Nor et al., 2001). Mice were then treated starting from day 12 until day 21 with _{D}(LPR) or control peptidomimetic (25 mg/Kg/day). Peptidomimetic compounds were injected daily intraperitoneally, and at the end of treatment the scaffolds were removed and the number of endothelial cells forming functional blood vessels determined. At day 21, all animals developed functional vessels with the expected cells density; these vessels were functional and positive for angiogenesis markers. The inventors observed a reduction of 36.7% in vessel formation in the group of animals treated with the _{D}(LPR) peptide compared to the animals treated with the control peptidomimetic (32.1 vessels per magnification field opposed to 20.3 in the presence of _{D}(LPR). (FIG. 1B) All together, these data show that the _{D}(LPR) peptidomimetic inhibits the formation and maturation of blood vessels in vivo, in two different angiogenesis animal model.

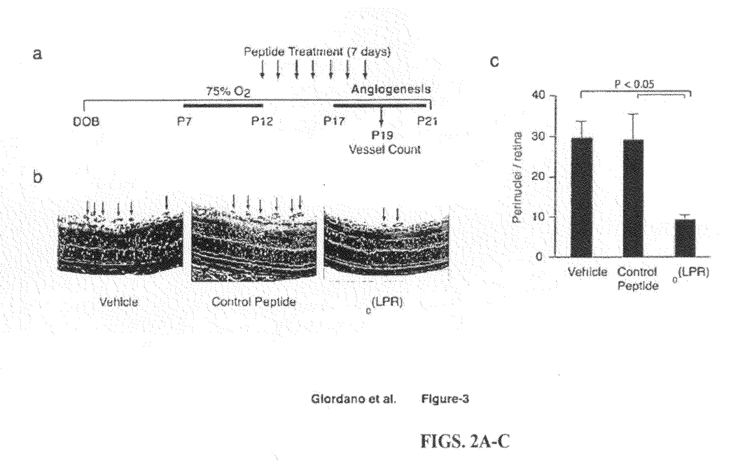

Studies were then carried out to address the question of whether the _{D}(LPR) peptidomimetic compound could be used as a drug to treat pathophysiological angiogenesis. For that purpose, animal models for two diseases were chosen in which angiogenesis is known to play an important role in disease progression: the retinopathy of prematurity (ROP) and cancer. For the ROP study, the mouse model of oxygen-induced retinopathy (Smith et al., 1994; Landenranta et al., 2001) was employed. Here, newly born mice (7-days old) were exposed to high levels of oxygen (75%) for 5 days and returned to room air (20.8% oxygen). The changes in oxygen levels induced a relative hypoxia in the animals to which the endothelial cells responded by activating oxygen-responsive elements, such as the hypoxia inducible factor-1 (HIF-1) and VEGF (Smith et al., 1994; Landenranta et al., 2001). Upon return to room-air (day 12), animal were then treated daily with _{D}(LPR) or control peptidomimetic for 7 days (20 mg/Kg). At the end of treatment (day 19), the eyes were enucleated and examined to determine the level of neovascularization by counting the number of vessels and endothelial cells protruding from the retina (FIG. 2A). A significant reduction in angiogenesis (68.5%) was observed in the animals treated with _{D}(LPR) compared to animal treated with vehicle only or control peptidomimetic (FIG. 2B, 2C) (29.8±3.8 nuclei/retina in the animal treated with vehicle only; 29.3±6.0 nuclei/retina in animal treated with control peptidomimetic; 9.4±1.0 nuclei/retina in animals treated with _{D}(LPR)).

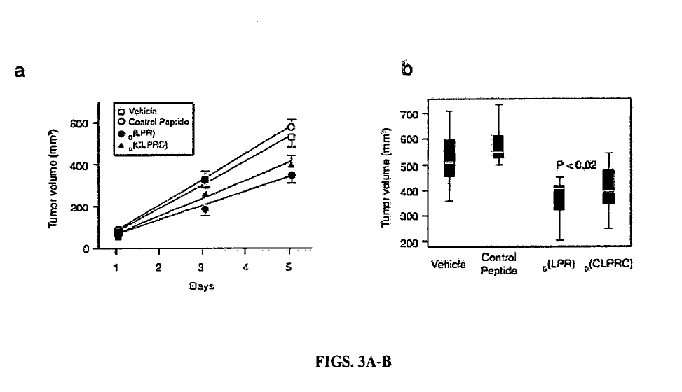

Next, studies were carred out to determine whether the _{D}(LPR) peptide would have an effect on tumor-induced neovascularization. For these studies, the highly angiogenic mouse model of breast cancer EF43.fgf4 (Deroanne et al., 1997; Hajitou et al, 1998) was chosen. In this model, the cancer cells produce and secrete fibroblast growth factor-4 (FGF-4), which induces the autocrine production of VEGF resulting in highly vascularized tumors. Animals were injected with the breast cancer cells subcutaneously in the mammary fat pad and tumor allowed to grow until they reached a small size (50-80 mm³). The animals bearing tumors were then treated daily with intraperitoneal injections of _{D}(LPR) (50 mg/Kg), control peptidomimetic or vehicle alone. After 5 days of treatment, a clear reduction in tumor volume was detected in the animals treated with _{D}(LPR) (FIG. 3). For these studies, the cyclic variant of the _{D}(LPR) peptidomimetic, _{D}(CLPRC), was employed. Animals bearing tumors treated with _{D}(CLPRC) (25 mg/Kg) also showed significant reduction in tumor volume (FIG. 3). No effect on tumor growth was observed in the animals treated with control peptidomimetic.

Taken together, the data show that _{D}(LPR) peptidomimetic and its cyclic version are a new class of angiogenesis inhibitor and targeting agent that should find important applications in the clinic as well as in later stage such as tube formation and maturation (SCID mouse model of human angiogensis). _{D}(LPR) when administered systemically in the mouse significantly reduce vessel formation during pathological angiogenesis (mouse model of ROP) as well as tumor induced-angiogenesis.

### Example 2

### Adipose Targeting/Obesity Studies

Diet and lifestyle contribute to the high incidence of obesity in the developed world. In the United States, approximately 65% of the adult population is overweight, with a body mass index of greater than or equal to 25 kg/m², and over 30% is obese (body mass index of greater than or equal to 30 kg/m²). Obesity is associated with increased risk for diabetes mellitus, cancer and heart disease, and it often causes shortening of human life. Advances in the treatment of obesity have thus far been rather limited with few drugs available to control abnormal fat accumulation (Clapham et al., 2001). Most anti-obesity agents are based on altering energy balance pathways and appetite by acting on receptors in the brain. Some drugs of this class (such as fenfluramine) have been withdrawn from the market due to unexpected toxicity. Recent attempts to develop compounds that inhibit absorption of fat through the gastrointestinal tract (such as orlistat, marketed under the trade name Xenical® by Roche) may improve antiobesity treatment. Still, even the most effective drugs can only reduce weight by up to 5%, and strict dieting is required for further weight loss (Clapham et al., 2001; Padwal & Majumdar, 2007).

It has been shown that non-neoplastic tissue growth (i.e., fat tissue) also depends on formation of new blood vessels (angiogenesis). For example, mice from different obesity models that received anti-angiogenic agents have shown a treatment dose-dependent reversible weight reduction and adipose tissue loss (Rupnick et al., 2002). These studies illustrate that adipose tissue mass is sensitive to angiogenesis inhibitors.

The present inventors thus carried out studies to assess the ability of the LPR VEGFR-1 targeting peptides of the present invention to target fat tissue and thereby reduce weight in diet-induced obese mice. To this end, diet-induced obese mice were divided in groups and treated with the VEGFR-1/NRP-1 targeting peptide. C57BL/6 ]-60% DIO mice (36-weeks old) were purchased from The Jackson Laboratory. These animals were fed on a high calorie diet (J-60%) to produce a diet-induced obesity (DIO) phenotype. Animals were divided in groups and treated daily with: [Group 1]_{D}(CLPRC) (SEQ ID NO:7) (N=5); [Group 2] CKGGRAKDC-GG-_{D}(KLAKLAK)₂ (SEQ ID NO:9) (N=3); [Group 3]_{D}(CLPRC) combined with CKGGRAKDC-GG-_{D}(KLAKLAK)₂ (SEQ ID NO:10) (N=4); [Group 4] vehicle only (N=4). All animals were hydrated by intraperitoneally administration of 1 ml of saline solution (0.9% sodium chloride solution, USP quality) 30 minutes prior to treatment. Mice were treated daily with _{D}(CLPRC) dissolved in phosphate buffered saline vehicle (PBS) (100 μl total injection) at a 50 mg/Kg/body weight dose (Groups 1 and 3); CKGGRAKDC-GG-_{D}(KLAKLAK)₂ (SEQ ID NO:9) in PBS was administered by subcutaneously (100 total volume) at 3 mg/Kg (Group 2) or at 1 mg/Kg when in combination with _{D}(CLPRC) (Group 3) 5 days a week (Monday thru Friday). Mice were weighted once a week.

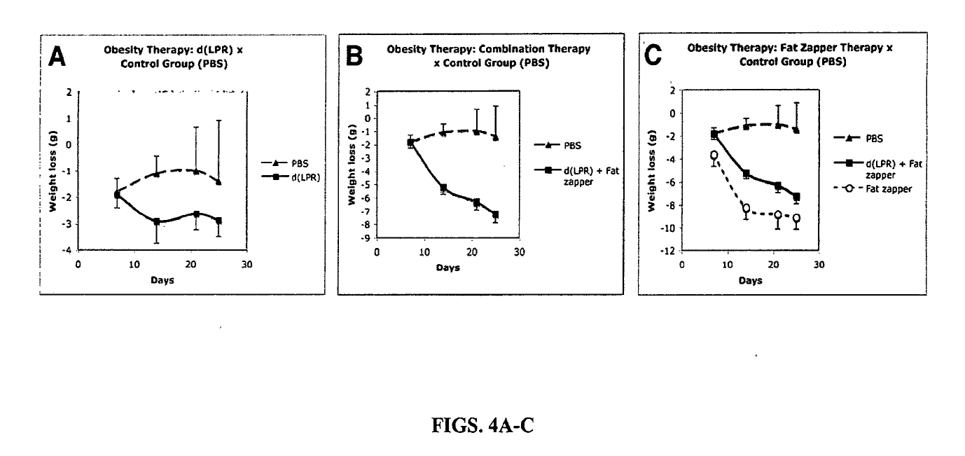

The results of the studies are shown in FIG. 4A-4C. In the group of obese mice treated with _{D}(CLPRC) injected daily intraperitoneally (50 mg/Kg), it can be seen that a fairly substantial weight loss of about 3 grams (approx. 6% of body weight) is observed over the treatment period, whereas the control mice showed a relative increase in weight (FIG. 4A). Next, to confirm the weight-loss effect of _{D}(CLPRC), a combination therapy experiment was designed. Obese mice were treated with the VEGFR-1/NRP-1 targeting peptide together with the fat-zapper anti-obesity compound. In previous studies, the inventors have shown that ablation of white fat could be achieved by selective targeting of the fat vasculature with the homing peptide CKGGRAKDC (SEQ ID NO:11) conjugated to the proapoptotic pepitde _{D}(KLAKLAK)₂ (SEQ ID NO:2) (Kolonin et al., 2004). This compound, named “Fat zapper” (sequence CKGGRAKDC-GG-_{D}(KLAKLAK)₂) (SEQ ID NO:9), induces fat ablation at therapeutic doses equal or higher than 3 mg/Kg/body weight. For the combination experiment, however, the VEGFR-1/NRP-1 targeting peptide _{D}(CLPRC) (SEQ ID NO:7) was combined with a sub-therapeutic dose of Fat-zapper (1 mg/Kg) with the expectation was that the anti-angiogenic effect of _{D}(CLPRC) would synergize with the tissue ablation effect of Fat-zapper.

Indeed, a significant weight loss of about 8 grams (up to 16% body weight) was observed when animals received both drugs (FIG. 4B). Animals that receive the combination therapy showed weight losses similar to the group of mice receiving fat-zapper alone at the optimal therapeutic dose of 3 mg/Kg (FIG. 4C), which lost about 10 grams (20% body weight). Taken together, these data whow that the VEGFR-1/NRP-1 targeting peptides induces weight loss in obese mice, and that they may synergize with other anti-obesity therapies. The VEGFR-1/NRP-1 target peptides may find important applications in the treatment of human obesity.

All of the compositions and methods disclosed and claimed herein can be made and executed without undue experimentation in light of the present disclosure. While the compositions and methods of this invention have been described in terms of preferred embodiments, it are apparent to those of skill in the art that variations maybe applied to the compositions and methods and in the steps or in the sequence of steps of the methods described herein without departing from the concept, spirit and scope of the invention. More specifically, it are apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,472,509
U.S. Pat. No. 5,021,236
U.S. Pat. No. 5,206,347
U.S. Pat. No. 5,401,511
U.S. Pat. No. 5,603,872
U.S. Pat. No. 5,889,155
U.S. Pat. No. 6,649,407
U.S. Patent Appln. 20040023415
Aase et al., Circulation, 104:358-364, 2001.
Arap et al., Science, 279:377-380, 1998.
Autiero et al., Nat. Med., 9:936-943, 2003.
Baichwal and Sugden, In: Gene Transfer, Kucherlapati (Ed.), Plenum Press, NY, 117-148, 1986.
Bakhshi et al., Cell, 41(3):899-906, 1985.
Barany and Merrifield, In: The Peptides, Gross and Meienhofer (Eds.), Academic Press, NY, 1-284, 1979.
Bellomo et al., Circ. Res., 86:E29-35, 2000.
Betsholtz et al., J. Physiol Heart Circ. Physiol., 290:H509-H511, 2006
Cardones and Banez, Curr. Pharm. Design, 12:387-394, 2006.
Carmeliet et al., Nat. Med., 7:575-583, 2001.
Carmeliet, Nature, 438:932-936, 2005.
Chorev and Goodman, Trends Biotechnol., 13:438-445, 1995.
Chorev, Biopolymers, 80:67-84, 2005.
Clapham, J. et al., Pharmacol. Ther. 89, 81-121, 2001.
Cleary and Sklar, Proc. Natl. Acad. Sci. USA, 82(21):7439-7443, 1985.
Cleary et al., J. Exp. Med., 164(1):315-320, 1986.
Coffin, In: Virology, Fields et al. (Eds.), Raven Press, NY, 1437-1500, 1990.
Deroanne et al., Cancer Res., 57:5590-5597, 1997.
Eliceiri and Cheresh, Curr. Opin. Cell Biol., 13(5):563-568, 2001.
Ellerby et al., Nature Med., 9:1032-1038, 1999.
Falciani et al., Chem. Biol., 12:417-426, 2005.
Ferrara, Endocr. Rev., 25:581-611, 2004.
Fischer, Curr. Protein Pept. Sci., 4:339-356, 2003.
Folkman, N. Engl. J. Med., 285:1182-1186, 1971.
Folkman, Nature Biotechnol., 15: 510, 1997.
Giordano et al., Nat. Med., 7:1249-1253, 2001.
Giordano et al., Chem. Biol., 12:1075-1083, 2005.
Gomez-Foix et al., J. Biol. Chem., 267:25129-25134, 1992.
Graham and Prevec, In: Methods in Molecular Biology: Gene Transfer and Expression Protocol, Murray (Ed.), Humana Press, Clifton, N.J., 7:109-128, 1991.
Grunhaus and Horwitz, Seminar in Virology, 3:237-252, 1992.
Hajitou et al., Oncogene, 17:2059-2071, 1998.
Hermonat and Muzycska, Proc. Natl. Acad. Sci. USA, 81:6466-6470, 1984.
Herz and Gerard, Proc. Natl. Acad. Sci. USA, 90:2812-2816, 1993.
Jackson et al., In: Goodman and Gilman's The Pharmacological basis of therapeutics, Hardman (Eds.), McGraw-Hill Medical Publishing Division, 809-841, 2001.
Jin et al., Nat. Med., 12:557-567, 2006.
Kaplan et al., Nature, 438:820-827, 2005.
Kolonin et al., Nat. Med., 6:625-632, 2004.
Landenranta et al., Proc. Natl. Acad. Sci. USA, 98:10368-10373, 2001.
Le Gal La Salle et al., Science, 259:988-990, 1993.
Levrero et al., Gene, 101:195-202, 1991.
Li et al., FASEB J., 20:1495-1497, 2006.
Luttun et al., Nat. Med., 8:831-840, 2002.
Luttun et al., Biochim. Biophys. Acta., 1654:79-94, 2004.
Mann et al., Cell, 33:153-159, 1983.
Merrifield, Science, 232(4748):341-347, 1986.
Murga et al., Blood, 105:1992-1999, 2005.
Nicolas and Rubinstein, In: Vectors: A survey of molecular cloning vectors and their uses, Rodriguez and Denhardt, eds., Stoneham: Butterworth, pp. 494-513, 1988.
Nicolau et al., Methods Enzymol., 149:157-176, 1987.
Nor et al., Lab. Invest., 81:453-463, 2001.
Olsson et al., Nat. Rev. Mol. Cell. Biol., 7:359-371, 2006.
Padwal and Majumdar, Lancet 369, 71-77, 2007.
Pan et al., Cancer Cell, 11:53-67, 2007.
Paskind et al., Virology, 67:242-248, 1975.
Physicians Desk Reference
Ragot et al., Nature, 361:647-650, 1993.
Remington's Pharmaceutical Sciences” 15th Edition, pages 1035-1038 and 1570-1580, 1990.
Remington's Pharmaceutical Sciences, 18th Ed. Mack Printing Company, pp. 1289-1329, 1990.
Ridgeway, In: Vectors: A Survey of Molecular Cloning Vectors and Their Uses, Rodriguez et al. (Eds.), Stoneham: Butterworth, 467-492, 1988.
Rosenfeld et al., Science, 252:431-434, 1991.
Rosenfeld, et al., Cell, 68:143-155, 1992.
Rupnick, et al., Proc. Natl. Acad. Sci. USA. 99, 10730-5, 2002.
Schneider and Sledge, Nat. Clin. Pract. Oncol., 4:181-189, 2007.
Smith et al., Invest. Ophthalmol. Vis. Sci., 35:101-111, 1994.
Soker et al., J. Cell Biochem., 85:357-368, 2002.
Stewart and Young, In: Solid Phase Peptide Synthesis, 2d. ed., Pierce Chemical Co., 1984.
Tam et al., J. Am. Chem. Soc., 105:6442, 1983.
Temin, In: Gene Transfer, Kucherlapati (Ed.), NY, Plenum Press, 149-188, 1986.
Tsujimoto and Croce, Proc. Natl. Acad. Sci. USA, 83(14):5214-5218, 1986.
Tsujimoto et al., Science, 228(4706):1440-1443, 1985.
Wang et al., J. Biol. Chem., 278:48848-48860, 2003.
Wong et al., Gene, 10:87-94, 1980.
Wu et al., Clin. Cancer Res,. 12:6573-6584, 2006.

1. An isolated peptide of 10 amino acids or less in size, comprising at least the contiguous amino acid sequence Leu Pro Arg.

2. The isolated peptide of claim 1, wherein said peptide is 7 amino acids or less in size.

3. (canceled)

4. The isolated of peptide of claim 1, further defined as comprising Cys Leu Pro Arg Cys (SEQ ID NO:1).

5. The isolated peptide of claim 4, further defined as consisting of SEQ ID NO:1

6. The isolated peptide of claim 5, further defined as a cyclic peptide.

7. The isolated peptide of claim 1, wherein the peptide consists of the sequence Leu Pro Arg or _{D}(Leu Pro Arg).

8. (canceled)

9. The isolated peptide of claim 1, further defined as comprising one or more D amino acids.

10. (canceled)

11. The isolated peptide of claim 1, wherein said peptide is attached to a molecule.

12. The isolated peptide of claim 11, wherein the molecule is a protein and the peptide is conjugated to the protein to form a protein conjugate.

13. The isolated peptide of claim 12, wherein the peptide is positioned at a terminus of the protein.

14. The isolated peptide of claim 11, wherein said molecule is a drug, a chemotherapeutic agent, a diagnostic agent, a radioisotope, a pro-apoptosis agent, an anti-angiogenic agent, a hormone, a cytokine, a growth factor, a cytotoxic agent, a peptide, a protein, an antibiotic, an antibody or a fragment or single chain antibody thereof, an imaging agent, survival factor, an anti-apoptotic agent, a hormone antagonist, or an antigen.

15. The isolated peptide of claim 14, wherein said molecule is a pro-apoptosis agent selected from the group consisting of gramicidin; magainin; mellitin; defensin; cecropin; (KLAKLAK)₂ (SEQ ID NO:2); (KLAKKLA)₂ (SEQ ID NO:3); (KAAKKAA)₂ (SEQ ID NO:4); (KLGKKLG)₃ (SEQ ID NO:5); Bcl-2; Bad; Bak; Bax; and Bik.

16. The isolated peptide of claim 14, wherein said molecule is an anti-angiogenic agent selected from the group consisting of thrombospondin, an angiostatin, pigment epithelium-derived factor, angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2, herbimycin A, PNU145156E, 16K prolactin fragment, Linomide, thalidomide, pentoxifylline, genistein, TNP-470, an endostatin, paclitaxel, Docetaxel, polyamines, a proteasome inhibitor, a kinase inhibitor, a signaling peptide, accutin, cidofovir, vincristine, bleomycin, AGM-1470, platelet factor 4, minocycline, endostatin XVIII, endostatin XV, the C-terminal hemopexin domain of matrix metalloproteinase-2, the kringle 5 domain of human plasminogen, a fusion protein of endostatin and angiostatin, a fusion protein of endostatin and the kringle 5 domain of human plasminogen, the monokine-induced by interferon-gamma (Mig), a fusion protein of Mig and IP10, soluble FLT-1 (fins-like tyrosine kinase 1 receptor), and kinase insert domain receptor (KDR).

17. The isolated peptide of claim 14, wherein said molecule is a cytokine selected from the group consisting of interleukin 1 (IL-1), IL-2, IL-5, IL-10, IL-11, IL-12, IL-18, interferon-γ (IF-γ), IF-γ, IF-β, a tumor necrosis factor, and GM-CSF (granulocyte macrophage colony stimulating factor).

18. (canceled)

19. The isolated peptide of claim 18, wherein said peptide is attached to a macromolecular complex that is a virus, a bacteriophage, a bacterium, a liposome, a microparticle, a magnetic bead, a yeast cell, or a mammalian cell.

20. The isolated peptide of claim 19, wherein said peptide is attached to a virus.

21. The isolated peptide of claim 20, wherein said virus is a lentivirus, papovavirus, adenovirus, retrovirus, AAV, vaccinia virus or herpes virus.

22. The isolated peptide of claim 1, wherein said peptide is attached to a solid support.

23. The isolated peptide of claim 22, wherein the solid support is a microtiter dish or microchip.

24. A protein fusion construct comprising the isolated peptide of claim 1 fused to a selected protein to form a protein fusion construct, wherein the protein fusion construct is not a naturally occurring protein.

25. The fusion construct of claim 24, wherein the selected protein is a pro-apoptosis agent, an anti-angiogenic agent, a hormone, a cytokine, a growth factor, a cytotoxic agent, a protein antibiotic, an antibody or fragment or single chain thereof, an anti-apoptotic agent, a hormone antagonist, or an antigen.

26. The fusion construct of claim 25, wherein said selected protein is a pro-apoptosis agent selected from the group consisting of gramicidin; magainin; mellitin; defensin; cecropin; (KLAKLAK)₂ (SEQ ID NO:2); (KLAKKLA)₂ (SEQ ID NO:3); (KAAKKAA)₂ (SEQ ID NO:4); (KLGKKLG)₃ (SEQ ID NO:5); Bcl-2; Bad; Bak; Bax; and Bik.

27. The fusion construct of claim 25, wherein said selected protein is an anti-angiogenic agent selected from the group consisting of thrombospondin, an angiostatin, pigment epithelium-derived factor, angiotensin, laminin peptides, fibronectin peptides, plasminogen activator inhibitors, tissue metalloproteinase inhibitors, interferons, interleukin 12, platelet factor 4, IP-10, 2-methoxyoestradiol, proliferin-related protein, angiopoietin 2, 16K prolactin fragment, endostatin, endostatin XVIII, endostatin XV, the C-terminal hemopexin domain of matrix metalloproteinase-2, the kringle 5 domain of human plasminogen, a fusion protein of endostatin and angiostatin, a fusion protein of endostatin and the kringle 5 domain of human plasminogen, the monokine-induced by interferon-gamma (Mig), a fusion protein of Mig and IP10, soluble FLT-1 (fins-like tyrosine kinase 1 receptor), and kinase insert domain receptor (KDR).

28. The fusion construct of claim 25, wherein said selected protein is a cytokine selected from the group consisting of interleukin 1 (IL-1), IL-2, IL-5, IL-10, IL-11, IL-12, IL-18, interferon-γ(IF-γ), IF-α, IF-β, a tumor necrosis factor, and GM-CSF (granulocyte macrophage colony stimulating factor).

29. A method of preparing a VEGFR-1/NRP-1 targeted construct comprising attaching the peptide of claim 1 to a molecule to prepare the construct.

30. A method of targeting the delivery of a molecule to cells that express VEGFR-1 or NRP-1, the method comprising:
contacting a cell population with a peptide of claim 11, wherein the cell population includes cells that express VEGFR-1 or NRP-1, wherein the molecule is delivered to said cells.

31. The method of claim 30, wherein the cells that express a VEGFR-1 or NRP-1 are in a subject, the peptide or protein fusion construct is formulated in a pharmaceutically acceptable composition and the composition is administered to the subject.

32. The method of claim 31, wherein the subject is a human subject.

33. The method of claim 30, wherein the method is further defined as a detection method and the method further comprises detecting the peptide or protein that has been delivered to the cells.

34. The method of claim 31, wherein subject has a disease or disorder and the method is further defined as a therapeutic method.

35. The method of claim 34, subject has a disease or disorder with an angiogensis component and the subject is in need of anti-angiogenesis therapy.

36. The method of claim 35, wherein the disease or disorder is a hyperproliferative disease, weight disorder, obesity, diabetes, asthma, arthritis, cirrhosis, or an ocular disease.

37. The method of claim 36, wherein the subject has a hyperoliferative disease

38. The method of claim 37, wherein the hyperproliferative disease is rheumatoid arthritis, inflammatory bowel disease, osteoarthritis, leiomyomas, adenomas, lipomas, hemangiomas, fibromas, vascular occlusion, restenosis, atherosclerosis, pre-neoplastic lesions (such as adenomatous hyperplasia and prostatic intraepithelial neoplasia), carcinoma in situ, oral hairy leukoplakia, or psoriasis.

39. The method of claim 37, wherein the hyperproliferative disease is a cancer.

40. The method of claim 39, wherein the cancer is selected from the group of cancers of the gum, tongue, lung, skin, liver, kidney, eye, brain, leukemia, mesothelioma, neuroblastoma, head, neck, breast, pancreatic, prostate, renal, bone, testicular, ovarian, mesothelioma, cervical, gastrointestinal, lymphoma, brain, colon, sarcoma and bladder.

41. The method of claim 36, wherein the subject has an ocular disease or disorder characterized by intraocular cellular proliferation or neovascularization.

42. The method of claim 41, wherein the ocular disease is selected from the group consisting of age-related macular degeneration, proliferative diabetic retinopathy, retinopathy of prematurity, glaucoma, and proliferative vitreoretinopathy.

43. The method of claim 34, wherein the subject has a weight disorder.
